(19) United States
(12) Patent Application Publication
(10) Pub. No.: US 2009/0105713 A1
(43) Pub. Date: Apr. 23, 2009

(54) Deployment handle for an implant deployment device

(75) Inventors: Lars S. Nimgaard, Koege (DK); Erik E. Rasmussen, Slagelse (DK)

Correspondence Address: COOK GROUP PATENT OFFICE, P.O. BOX 2269, BLOOMINGTON, IN, 47402, US

(73) Assignees: William Cook, Europe ApS, Bjaeverskov (DK); Cook Incorporated, Bloomington, IN (US)

(21) Appl. No.: 12/287,393

(22) Filed: Oct. 9, 2008

Related U.S. Application Data
(60) Provisional application No. 60/998,098, filed on Oct. 9, 2007.

(51) Int. Cl. A61B 17/58; A61B 17/00

(52) U.S. Cl. 606/99; 606/207

(57) ABSTRACT

A deployment handle for an implant deployment device (100) facilitates withdrawal of a sheath (32). The sheath (32) is gripped between a first cam cleat (122) and a first ledge (124) and a second cam cleat (126) and a second ledge (128). The first cam cleat (122) and the first ledge (124) are attached to a carriage (120), which moves in a withdrawal direction upon actuation of a lever (130). During movement of the carriage (120) in the withdrawal direction, the first cam cleat (122) grips the sheath (32) or a sheath pulling member (118) thus causing movement of the sheath (32) in the withdrawal direction. During this movement, the second cam cleat (126) releases its grip upon the sheath (32) or a sheath pulling member (118). Upon release of the lever (130), the carriage (120) moves in a direction opposite to the withdrawal direction by means of a helical spring (140). During this movement, the second cam cleat (126) grips the sheath (32) or the sheath pulling member (118) whilst the first cam cleat (122) releases its grip upon the sheath (32) or the sheath pulling member (118). The deployment handle (100) allows a controlled, step-wise withdrawal of a sheath (32) covering an implant (18).

### CROSS-REFERENCE TO RELATED APPLICATIONS

This application claims priority of provisional application Ser. No. 60/998,098, filed Oct. 9, 2007.

### TECHNICAL FIELD

The present invention relates to a deployment handle for an implant deployment device, for moving a component of an implant deployment device. The present invention further relates to an implant deployment assembly.

### BACKGROUND OF THE INVENTION

The use of delivery devices employing catheters has long been known for a variety of medical procedures, including procedures for establishing, re-establishing or maintaining passages, cavities or lumens in vessels, organs or ducts in human and veterinary patients, occlusion of such vessels, delivering medical treatments, and other interventions. For these procedures, it has also long been known to deliver an implant by means of a catheter, often intraluminally. For example, a stent, stent-graft, filter or occlusion device may be delivered intraluminally from the femoral artery for deployment.

For procedures in which a prosthesis or other device is implanted into a patient, the device to be implanted is normally held onto the catheter in a compressed state and then released from the catheter so as to expand to its normal operating state, prior to withdrawal of the catheter from the patient to leave the implant in position.

A variety of delivery mechanisms is known in the art. These generally involve positioning the implant on a distal part of a delivery device, that is, at an end furthest from the external manipulation end used by the clinician during the deployment procedure. The prosthesis or implant is normally held to the distal end of the catheter by a suitable restraining mechanism, restraining wires being just one known example. It is also conventional to cover the implant with a sheath in order to protect the implant and also the patient's lumens or organs during the delivery process. Once the implant has been positioned at the location in which it is to be released, the sheath is retracted along the catheter to expose the implant. The implant is then expanded, either automatically, if the implant is of the self-expanding type, or by a suitable expanding mechanism if not, such as by means of an expansion balloon.

In cases where a sheath or other covering is provided, some delivery devices include a mechanism by which the sheath can be withdrawn by being pulled back towards the external manipulation end of the delivery device, that is, towards the surgeon or other clinician. The force required to withdraw such a sheath may be very large. Furthermore, the resistance to withdrawal of a sheath may vary, which can cause problems for a controlled and safe uncovering of an implant.

The sheath may be withdrawn by the surgeon or clinician gripping the proximal end of the sheath with one hand, and the catheter with the other hand, and pulling back the sheath relative to the catheter. This method is not only hard work, but also the surgeon or clinician is unable to exert much control over the withdrawal process. Moreover, use of such force to withdraw a sheath may result in shifting of the previously carefully placed implant.

U.S. Pat. No. 6,402,760 discloses a device for withdrawing a telescopic sheath that takes advantage of a lever mechanism to convert a small force exerted on the lever into a large force for withdrawing the sheath. The sheath is attached to a moveable carriage. Lifting up of the lever causes the carriage to move in a withdrawal direction and thus withdraw the sheath. Whilst this device reduces the force required to withdraw a sheath, the range of movement is limited by the action of the lever. Furthermore, this device must be designed for each type of sheath since the available movement of the carriage must correspond at least to the desired extent of withdrawal of the sheath. Pressing the lever down would cause the carriage (and thus any attached sheath) to move back into the patient. Therefore only a single lifting of the lever is possible and this must result in the full desired withdrawal of the sheath.

### SUMMARY OF THE INVENTION

The present invention seeks to provide an improved deployment handle for an implant deployment device and an improved implant deployment assembly.

According to a first aspect of the present invention there is provided a deployment handle for moving a component of an implant deployment device, including: a withdrawal member able to be coupled to a component to be moved, and an actuator able to move towards a withdrawal axis of the handle, wherein movement of the actuator in a direction towards the axis actuates the withdrawal member, thereby to move a component in a withdrawal direction.

According to a second aspect of the present invention there is provided a deployment handle for moving a component of an implant deployment device, including: a housing, a withdrawal member provided in the housing, and an actuation member coupled to the withdrawal member; wherein the actuation member is movable in a direction towards the housing to actuate the withdrawal member in a direction substantially parallel to a longitudinal axis of an implant withdrawal member.

Actuation of the withdrawal member by moving the actuation member in a direction towards the housing enables a user to take advantage of a squeezing action between the actuation member and the deployment handle to effect withdrawal of a component of an implant deployment device. Such an action enables the user to use only a single hand to use the deployment handle and thus withdraw the desired component.

Preferably, the withdrawal member is movable and movement of the actuator in a direction towards the axis results in movement of the withdrawal member in a withdrawal direction. In this way, a component connected to the withdrawal member is also moved in the withdrawal direction.

In an embodiment, movement of the actuator in a direction away from the axis results in movement of the withdrawal member in a direction opposite to the withdrawal direction. This allows the withdrawal member to return to its original position prior to effecting further withdrawal of a component.

The withdrawal member is preferably able to move in a withdrawal direction from a first position to a second position, and is preferably biased into its first position. This means that the user does not have to exert any effort to return the withdrawal member to its first position.

Preferably the actuator has an actuation position wherein it is towards the axis, and a release position, wherein it is away from the axis, and wherein the actuator is biased into the release position. In the preferred embodiment, the withdrawal member and the actuator are arranged such that the withdrawal member is biased into its first position and the actuator is biased into its release position. This arrangement means that effort is only required to cause the withdrawal member to move in the withdrawal direction.

The withdrawal member may include a first gripping element. Preferably the withdrawal member is movable and the first gripping element is operable to grip a component during movement of the movable withdrawal member in a withdrawal direction and to release its grip on a component during movement of the withdrawal member in a direction opposite to the withdrawal direction. This arrangement allows the withdrawal member to withdraw the component in several small steps.

The deployment handle preferably includes a second gripping element for gripping a component to be moved, wherein the second gripping element is operable to grip a component to be moved during release of the component to be moved by the first gripping element. This ensures that movement of the withdrawal member in a direction opposite to the withdrawal direction does not cause any movement of the component in a direction opposite to the withdrawal direction.

According to a third aspect of the present invention there is provided a deployment handle for moving a component of an implant deployment device, including: a movable withdrawal member and a first gripping element for gripping a component to be moved, wherein the first gripping element is provided on the movable withdrawal member, and wherein the first gripping element is operable to grip a component during movement of the movable withdrawal member in a withdrawal direction and to release its grip on a component during movement of the withdrawal member in a direction opposite to the withdrawal direction.

Provision of a gripping element that is able to release its grip on a component during movement of the withdrawal member in a direction opposite to the withdrawal direction allows the withdrawal member to effect withdrawal of the component in several small steps. This allows greater control of the withdrawal process.

According to a fourth aspect of the present invention, there is provided a deployment handle for withdrawing a component of an implant deployment device including: a moveable withdrawal member able to be coupled to a component to be withdrawn; a first gripping element for gripping a component to be withdrawn, wherein the first gripping element is provided on the moveable withdrawal member, and wherein the first gripping element is operable to grip a component during movement of the moveable withdrawal member in a withdrawal direction and to release its grip on a component during movement of the withdrawal member in a direction opposite to the withdrawal direction, wherein the first gripping element is biased into a gripping state; a second gripping element for gripping a component to be moved, wherein the second gripping element is operable to grip a component to be moved during release of the component to be moved by the first gripping element, wherein the second gripping element is biased into a gripping state; an actuator able to move towards a withdrawal axis of the handle, wherein movement of the actuator in a direction towards the axis actuates the withdrawal member resulting in movement of the withdrawal member in a withdrawal direction from a first position to a second position, thereby to withdraw a component, wherein the actuator has an actuation position, wherein it is towards the axis, and a release position, wherein it is away from the axis, and wherein the actuator is biased into the release position and wherein the withdrawal member is biased into its first position.

According to a fifth aspect of the present invention there is provided a kit including an implant deployment device including a component to be moved, and a deployment handle as described above.

The implant deployment device in the kit may include a component to be withdrawn. The component to be moved may be a sheath for covering an implant to be deployed.

### BRIEF DESCRIPTION OF THE DRAWING

Embodiments of the present invention are described below, by way of example only, with reference to the accompanying drawings, in which:

FIGS. 1 and 2 show an example of a deployment device;

FIG. 3 shows in cross-section a distal end of a catheter and a deployment handle, with a sheath in a partially withdrawn condition;

FIG. 4 shows a deployment handle according to an embodiment of the present invention coupled to an implant deployment device;

FIG. 5 shows an exploded view of the deployment handle of FIGS. 3 and 4;

FIG. 6 shows a cross-section of an embodiment of the deployment handle of FIGS. 3 and 4 in the non-actuated configuration;

FIG. 7 shows a cross-section of the deployment handle of FIGS. 3 and 4 in the actuated configuration; and

FIGS. 8 and 9 show another embodiment of a deployment handle.

### DETAILED DESCRIPTION

It is to be understood that the Figures are schematic and do not show the various components in their actual scale. In many instances, the Figures show scaled up components to assist the reader.

In this description, when referring to a deployment assembly, the term distal is used to refer to an end of a component which in use is furthest from the surgeon during the medical procedure, including within a patient. The term proximal is used to refer to an end of a component closest to the surgeon and in practice in or adjacent an external manipulation part of the deployment or treatment apparatus.

On the other hand, when referring to an implant such as a stent or stent graft, the term proximal refers to a location that in use is closest to the patient's heart, in the case of a vascular implant, and the term distal refers to a location furthest from the patient's heart.

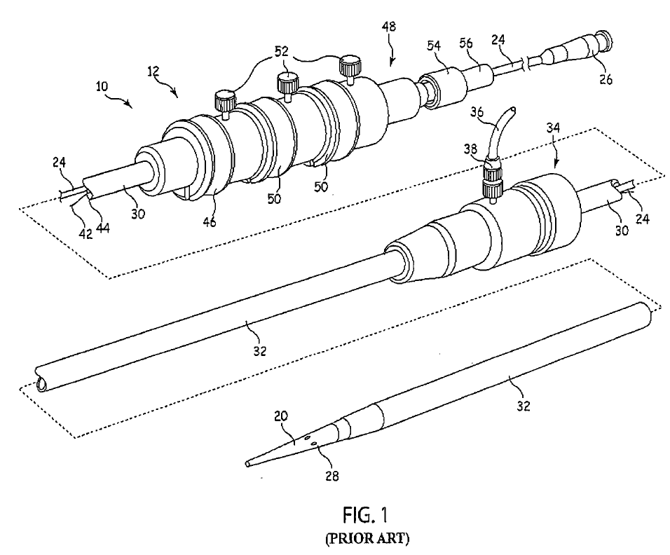

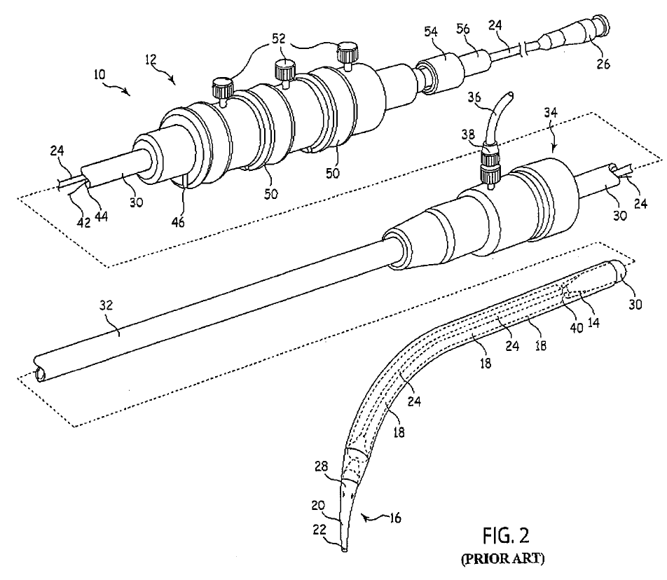

Referring to FIGS. 1 and 2, a known type of introducer 10 includes an external manipulation section 12, a proximal attachment region 14 and a distal attachment region 16. The proximal attachment region 14 and the distal attachment region 16 secure the two ends of the implant 18. During the medical procedure to deploy the implant 18, the proximal and distal attachment regions 14 and 16 will travel through the patient's vasculature, in this example, to a desired deployment site. The external manipulation section 12 at the proximal end of the assembly 10, which is operated by a surgeon to manipulate the introducer, remains outside of the patient throughout the procedure.

The distal attachment region 16 of the introducer 10 includes a dilator tip 20, which is typically provided with a bore 22 therein for receiving a guide wire (not shown) of conventional type. The longitudinal bore 22 also provides a channel for the introduction of medical reagents. For example, it may be desirable to supply a contrast agent to allow angiography to be performed during placement and deployment phases of the medical procedure.

An inner catheter or cannula 24, conventionally made from a flexible thin walled metal tube, is fastened to the dilator tip 20. The inner catheter 24 is flexible so that the introducer 10 can be advanced along a relatively tortuous vessel, such as a femoral artery, and so that the distal end of the assembly 10 can be longitudinally and rotationally manipulated. The inner catheter 24 carries a stent 18 or other device to be implanted in the patient. The inner catheter 24 extends through the introducer 10 to the manipulation section 12, terminating at a connection device 26, in conventional manner.

The connection device 26 is designed to accept a syringe to facilitate the introduction of reagents into the inner catheter 24 and for this purpose is typically provided with a threaded luer lock connection.

Where provided, a pusher sheath or rod 30 (hereinafter referred to as a pusher member), typically made from a plastics material, is mounted coaxially with and radially outside of the inner catheter 24. The pusher member 30 is “thick walled”, that is the thickness of its wall is preferably several times greater than that of the inner catheter 24. In some instances, the pusher member 30 and the inner catheter 24 are the same component, possibly having different outer diameters at the location at which the stent 18 is to be carried.

A sheath 32 extends coaxially over and radially outside of the pusher member 30. The pusher member 30 and the sheath 32 extend distally to the manipulation region 12.

The implant 18, which may be a stent, a stent-graft or any other implant or prosthesis deliverable by the introducer 10, is retained in a compressed condition by the sheath 32. The sheath 32 extends proximally to a sheath manipulator and haemostatic sealing unit 34 of the external manipulation section 12. The haemostatic sealing unit 34 includes a haemostatic seal (not shown) and a side tube 36 held to the unit 34 by a conventional luer lock 38.

The sheath manipulator and haemostatic sealing unit 34 also includes a clamping collar (not shown) that clamps the sheath 32 to the haemostatic seal and a silicone seal ring (not shown) that forms a haemostatic seal around the pusher rod 30. The side tube 38 facilitates the introduction of medical fluids between the pusher member 30 and the sheath 32. Saline solution is typically used.

During assembly of the introducer 10, the sheath 32 is advanced over the proximal end of the dilator tip 20 of the distal attachment region 16 while the implant 18 is held in a compressed state by an external force. A suitable distal attachment (retention) section (not visible in this view) is coupled to the pusher member 30 and retains a distal end 40 of the implant 18 during the procedure. The distal end of the implant 18 may be provided with a loop of material (not shown) through which a distal restraining wire 42 extends. The distal restraining wire 42 also extends through an aperture (not shown in FIGS. 1 and 2) in the proximal attachment region 14 into an annular region 44 between the inner catheter 24 and the pusher member 30. The distal restraining wire 42 extends through the annular space 44 to the manipulation region 12 and exits the annular space 44 at a distal wire release mechanism 46.

A proximal portion of the external manipulation section 12 includes at least one release wire actuation section 50 mounted on a body 48, in turn mounted onto the pusher member 30. The inner catheter 24 passes through the body 48. The distal wire release mechanism 46 and the proximal wire release mechanism 50 are mounted for slidable movement on the body 48.

The positioning of the proximal and distal wire release mechanisms 46 and 50 is such that the proximal wire release mechanism or mechanisms 50 must be moved before the distal wire release mechanism 46 can be moved, such that the proximal end of the implant, that is the end of the implant which will be upstream in the direction of fluid flow in the patient's vasculature, is released first. Therefore, the distal end of the implant 18 cannot be released until a self-expanding zigzag stent thereof has been released. Clamping screws 52 prevent inadvertent early release of the implant 18. A haemostatic seal (not shown) is included so that the restraining wires can extend out through the body 48 without unnecessary blood loss during the medical procedure.

A proximal portion of the external manipulation section 12 includes a pin vise 54 mounted onto the proximal end of the body 48. The pin vise 54 has a screw cap 56. When screwed in, vise jaws (not shown) of the pin vise 54 clamp against or engage the inner catheter 24. When the vise jaws are engaged, the inner catheter 24 can only move with the body 48 and hence it can only move with the pusher member 30. With the screw cap 56 tightened, the entire assembly can be moved together as one piece.

Once the introducer 10 is in the desired deployment position, the sheath 32 is withdrawn to just distal of the proximal attachment section 14. This action releases the middle portion of the implant 18, in this example a stent or stent-graft, so that it can expand radially. Consequently, the stent or stent-graft 18 can still be rotated or lengthened or shortened for accurate positioning. The proximal end of the self-expanding stent however, is still retained at the dilator tip 20 by means of the restraining wires. Also, the distal end of the stent or stent-graft 18 is still retained within the sheath 32.

Next, the pin vise 54 is released to allow small movements of the inner catheter 24 with respect to the pusher member 30 to allow the stent or stent-graft 18 to be lengthened, shortened, rotated or compressed for accurate placement in the desired location within the lumen. X-ray opaque markers (not shown) may be placed along the stent or stent-graft 18 to assist with placement of the prosthesis.

When the proximal end of the stent or stent-graft 18 is in place, the proximal restraining wire (not shown) is withdrawn by movement of the proximal wire release mechanism 50. The proximal wire release mechanism 50 and the proximal restraining wire can be completely removed by passing the proximal wire release mechanism 50 over the pin vise 54, the screw cap 56 and the connection unit 26.

Next, the screw cap 56 of the pin vise 54 is loosened, after which the inner catheter 24 can be pushed in a distal direction, that is towards the inside of the patient, so as to move the dilator tip 20 in a distal direction. This fully releases the proximal end of the stent or stent-graft 18, allowing it to expand so as to engage the lumen walls of the artery or vein. From this stage on, the proximal end of the stent or stent-graft 18 cannot be moved again.

Once the proximal end of the stent or stent-graft 18 is anchored, the sheath 32 is withdrawn distally of the proximal attachment section 14, which withdrawal allows the distal end of the stent or stent-graft 18 to expand. Until this point and in particular until the distal wire release mechanism 46 is actuated to release the distal restraining wires from the distal end of the stent 18, the distal end 40 may still be repositioned as needed.

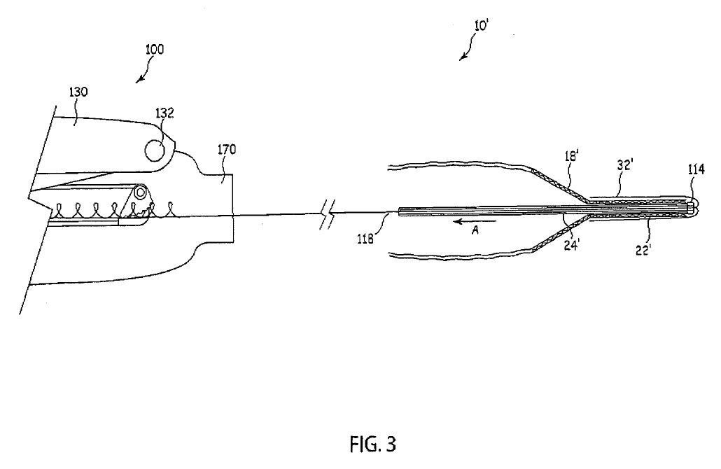

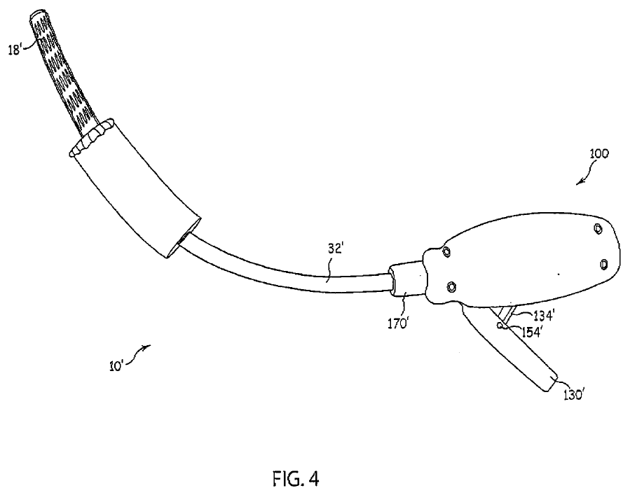

Copending U.S. patent application Ser. No. 60/861,860 (published as US 2008/0132879), the contents of which are hereby incorporated by reference, describes an implant deployment device 10′ for deploying stents, stent-grafts and other implants into a patient, with which the deployment handle described in the present application is particularly envisaged to be used. This is illustrated in FIGS. 3 and 4. The implant deployment device includes a catheter 24′ with a containment sheath 32′ in place of a conventional sheath 32 and conventional restraining wires. Held onto the catheter 24′ is a stent-graft structure 18′. The containment sheath 32′ extends over the entirety of the stent-graft section of the stent-graft structure 18′ so as to constrain it in its entirety on the catheter 24′, until the sheath 32′ is removed. The containment sheath 32′ extends from the distal position of the stent graft section to the tip and then into the central lumen 22′ of the catheter 24′. It may extend throughout the lumen to the proximal end of the implant deployment device 10′, in other words to the external manipulation section of the implant deployment device 10′. The sheath 32′ is withdrawn by pulling a pulling member 118′ through the central lumen 22′ in the catheter 24′ towards the external manipulation section of the implant deployment device 10′. As this is effected, the containment sheath 32′ is pulled over the end of the catheter 24′ and into the central lumen 22′ of the catheter 24′. As this occurs the containment sheath 32′ is, in effect, withdrawn into the catheter 24′, thereby releasing the stent-graft section gradually from its distal end to its proximal end. Continued pulling will gradually pull the entirety of the containment sheath 32′ into the open end of the catheter 24′, thereby releasing the entirety of the stent-graft section.

FIGS. 3 and 4 illustrate an embodiment of a deployment handle being used with the implant deployment device described in US 2008/0132879. The cross-sectional view of FIG. 3 shows the sheath 32′ having been partially withdrawn from its location over the stent-graft section of the stent-graft structure 18′. The pulling member 118 is pulled in the direction of the arrow A, that is, towards the external manipulation section of the delivery device. As a pulling action in direction A is effected, the containment sheath 32′ is pulled over the end 114 of the catheter 24′ and into the central lumen 22′ of the catheter 24′. As this occurs the containment sheath 32′ is in effect withdrawn into the catheter 24′, thereby releasing the stent-graft section of the stent-graft structure 18′ gradually. This arrangement is particularly advantageous for prostheses that include one portion which is not compressed onto the delivery catheter, or any other prosthesis which benefits from being expanded from its distal end first.

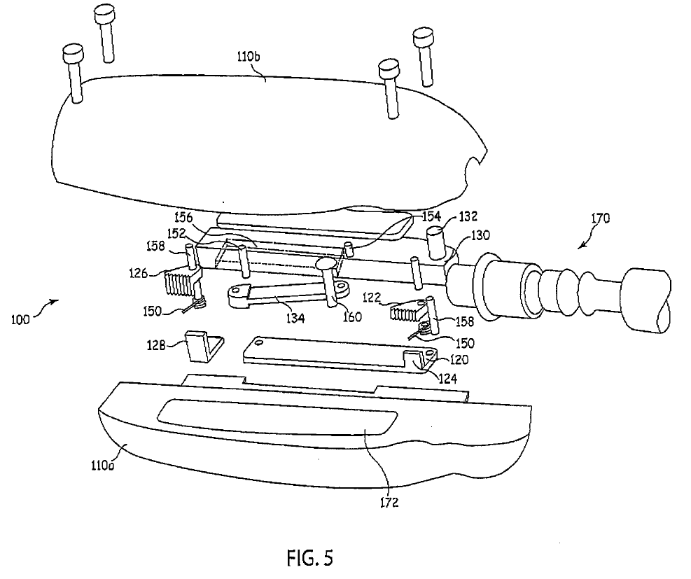

Referring now in particular to FIG. 5, which shows an exploded view of the components making up a deployment handle 100, a preferred embodiment of the invention is now described.

The deployment handle 100 includes a deployment handle body 110, which is formed of two parts, a “lower” part 110a and an “upper” part 110b. Together these form an outer casing for the working components of the deployment handle 100 having an overall size and shape suited to be handheld. A guide channel 172 is formed in the internal surface of the lower part of the deployment handle body 110a, and extends along the longitudinal axis of the deployment handle body 110. A carriage 120 is located in the guide channel 172 and is held therein by means of a bolt 160. The carriage 120 has a generally rectangular shape and is provided with a first ledge 124 at the distal end thereof. The first ledge 124 extends perpendicularly from an edge of the carriage 120. A first cam cleat 122 is located on the carriage 120. The first cam cleat 122 is located adjacent the ledge 124 by means of a cam cleat pivot 158. The cam cleat 122 is located such that its teeth are positioned facing the perpendicularly extending first ledge 124. A coiled torsion spring 150 is also located on the cam cleat pivot 158.

A second ledge 128 is fixed onto the internal surface of the lower part of the deployment handle body 110a so that it extends perpendicularly in a similar manner to the first ledge 124. The second ledge 128 is located towards the proximal end of the deployment handle body 100 and on the same longitudinal axis as the first ledge 124. Adjacent the second ledge 128 is located a second cam cleat 126. This is provided on a cam cleat pivot 158 along with a spring 150. The teeth of the second cam cleat 126 face the second ledge 128. The arrangement of the second ledge 128 and the second cam cleat 126 is very similar to that of the first ledge 124 and the first cam cleat 122. However, the first cam cleat 122 and the first ledge 124 are provided on the carriage 120, whereas the second cam cleat 126 and the second ledge 128 are provided directly on the inner surface of the lower part of the deployment handle body 110a.

At the proximal end of the carriage 120, a pivot 152 connects the carriage 120 to a proximal (lower) end of an actuation strut 134. The distal (upper) end of the actuation strut 134 is connected by means of a pivot 154 to an actuation lever 130. The pivot 154 connecting the actuation strut 134 to the actuation lever 130 is located, in this embodiment, approximately one-third along the length of the actuation lever 130 from its distal end. At the distal end of the actuation lever 130 is provided a lever pivot 132 for pivotably connecting the distal end of the actuation lever 130 to the internal surface of at least one of the parts 110a, 110b of the deployment handle body 110. A slot 156 is provided in a surface of the actuation lever 130 adjacent the actuation strut 134.

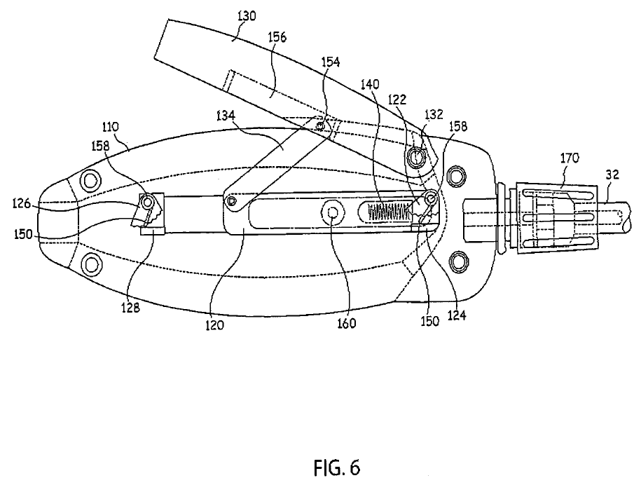

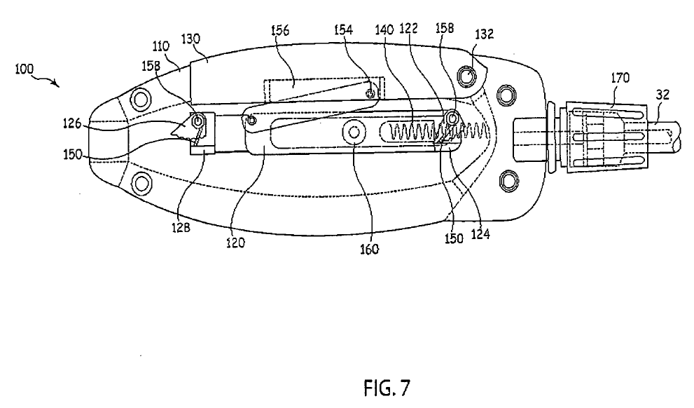

Referring now also to FIGS. 6 and 7, the deployment handle 100 is able to be connected to an implant deployment device 10, by means of connection elements 170, so that it sits over the inner catheter 24; 24′ and the sheath 32; 32′.

The carriage is able to translate along a longitudinal axis of the body 110 in the guide channel 172 from a distal position (shown in FIG. 6) to a proximal position (shown in FIG. 7). The actuation lever 130 is able to move away from, and towards, the body 110 of the deployment handle 100 around the lever pivot 132. The actuation lever 130, the actuation strut 134 and the carriage 120 are articulated such that movement of the actuation lever 130 towards the body 110 of the deployment handle 100 causes movement of the carriage 120 from its distal position (FIG. 6) to its proximal position (FIG. 7). Movement of the actuation lever 130 away from the body 110 of the deployment handle 100 causes movement of the carriage 120 from its proximal position (FIG. 7) to its distal position (FIG. 6). It can thus be seen that “up and down” motion of the actuation lever 130 (between a “release” position (FIG. 6) and an “actuation” position (FIG. 7)) causes “back and forth” movement of the carriage 120 by means of the actuation strut 134.

The carriage 120 is biased into its distal position (as shown in FIG. 6) by means of a helical spring 140. The spring 140 is affixed to the deployment handle body 110 at its distal end and to the carriage 120 at its proximal end. Movement of the carriage 120 thus causes tension and relaxation of the spring 140. The helical spring 140 is in a relaxed configuration when the carriage 120 is in its proximal position (FIG. 6), but is under tension when the carriage 120 is in its proximal position (FIG. 7). The biasing of the carriage 120 into the distal position, and the articulations between the carriage 120, the actuation lever 130 and the body 110 of the deployment handle 100, result in the actuation lever 130 being biased into its release position (i.e. away from the body 110 of the deployment handle 100). It can thus be seen that pressure on the actuation lever 130 is required, in this embodiment, for the carriage 120 to remain in its proximal position (as shown in FIG. 7).

As indicated above, the first cam cleat 122 is oriented such that its teeth face the first ledge 124. The teeth of the first cam cleat 122 thus enable gripping of a component located between the teeth and the first ledge 124. The first cam cleat 122, which is pivotably mounted to the carriage 120, is biased by means of the spring 150 into a position whereby the cam cleat 122 is urged in an anticlockwise direction (as viewed in FIGS. 5 to 7). This is referred to hereinafter as the “closed” position.

In a similar manner, the second cam cleat 126 is pivotably mounted adjacent an opposed second ledge 128, fixed to the body 110 of the deployment handle 100. Similarly to the arrangement of the cam cleat 122 with the first ledge 124, the second cam cleat 126 is biased by means of a spring 150 into a position in an anticlockwise direction (as viewed in FIGS. 5 to 7) (i.e. a “closed” position).

Use of the deployment handle 100 to withdraw a sheath 32; 32′ from a stent deployment device 10; 10′, such as that illustrated in FIGS. 3 and 4, is now described.

It can be seen from the above-described arrangement (illustrated in FIGS. 6 and 7) that since the first cam cleat 122 and the second cam cleat 126 are biased into a closed configuration, a gripping function between the first cam cleat 122 and the first ledge 124 and between the second cam cleat 126 and the second ledge 128 is provided in the absence of any other force on the cam cleats 122, 126. However, when a small amount of effort is provided in a direction against the bias of the cam cleats 122, 126, rotation of each cam cleat 122, 126 in a clockwise direction (as illustrated in FIGS. 5 to 7) is possible. Such clockwise rotation causes release of the gripping function.

In use, the deployment handle 100 is coupled to a stent deployment device 10′, such as that described in US 2008/0132879, by means of connection elements 170. A pulling member 118 attached to a sheath 32′ to be removed from a stent is threaded between the first cam cleat 122 and the first ledge 124 and between the second cam cleat 126 and the second ledge 128. Once threaded between the cam cleats 122, 126 and the ledges 124, 128, the bias towards the closed position of the cam cleats 122, 126 provides gripping of the pulling member 118.

In order to withdraw the sheath, the surgeon or clinician squeezes the actuation lever 130 and the body 110 of the deployment handle 100 together. As can be seen most clearly in FIGS. 6 and 7, squeezing together of the actuation lever 130 and the actuation body 110 causes the distal end of the actuation strut to move downwards and the proximal end of the actuation strut 134 to move in a proximal direction. The proximal end of the actuation strut 134 thus pulls the carriage 120 along the guide channel 172 by 1 to 2 cm in a proximal direction. Therefore, this squeezing action results in movement of the carriage 120 from its distal position (FIG. 6) to its proximal position (FIG. 7). Of course, the skilled person would appreciate that the distance traveled by the carriage 120 can be altered by adjusting the arrangement of the actuation lever 130, the strut 134 and the carriage 120.

As the carriage 120 moves proximally, the bias of the first cam cleat 122 to its closed position ensures that the pulling member 118 is gripped between the first cam cleat 122 and the first ledge 124 thus drawing the pulling member 118 along with movement of the carriage 120 in the proximal direction. At the same time, this movement of the pulling member 118 exerts a small force on the second cam cleat 126 in the proximal direction. This causes the second cam cleat 126 to rotate in a clockwise direction thereby releasing its grip on the pulling member 118. It can be seen, therefore, that squeezing of the actuation lever 130 towards the body 110 of the deployment handle 100 enables the sheath 32 to be withdrawn by a distance up to the maximum distance of movement of the carriage 120 (i.e. in practice, approximately 1 to 2 cm.

When the surgeon or clinician releases their grip on the actuation lever 130, the spring 140 causes the carriage 120 to move from its proximal position (FIG. 7) to its distal position (FIG. 6). Since the pulling member 118 is no longer being pulled in the proximal direction and thus is no longer exerting an effort on the second cam cleat 126, the second cam cleat 126, biased into its closed position, grips the pulling member 118. This avoids any reverse movement of the pulling member 118 in the distal direction as the carriage 120 moves from its proximal position (FIG. 7) to its distal position (FIG. 6). Indeed, any movement of the pulling member 118 towards the distal direction would result in an even tighter grip of the pulling member 118 between the second cam cleat 126 and the second ledge 128. This gripping by the second cam cleat 126 along with movement of the carriage 120 in the distal direction causes a small force to be exerted on the first cam cleat 122 by the pulling member 118. This causes a rotation in a clockwise direction of the first cam cleat 122. As this occurs, the grip of the first cam cleat 122 on the pulling member 118 is released, and the teeth are able to slide over the pulling member 118 in the distal direction until the carriage 120 has once again reached its distal position.

After release of the actuation lever 130, the surgeon or clinician repeats the process until the stent-graft structure 18 is fully exposed and able to expand. It can be seen, therefore, that the above-described deployment handle provides for well controlled, step-wise withdrawal of a sheath 32. Furthermore, since movement of the sheath in the withdrawal direction is provided by squeezing together of the actuation lever 130 and the body 110 of the deployment handle 100, and because the actuation lever 130 is biased towards its open position (as shown in FIG. 6), withdrawal of the sheath 32 can be effected by the surgeon or clinician using a single hand, freeing the other hand to guide or hold the delivery assembly.

The skilled person will appreciate that the above-described embodiment is merely exemplary, and other arrangements can be envisaged. Modifications, therefore, may be made thereto without departing from the scope of the attached claims.

Whilst the above-described deployment handle is particularly envisaged for use with the stent deployment device 10′ described in US 2008/0132879, the skilled person will appreciate that it could be modified to facilitate withdrawal of a conventional sheath 32 from an implant deployment device 10 such as that illustrated in FIGS. 1 and 2. With reference to FIGS. 1 and 2, the deployment handle 100 could be fitted to a stent deployment device 10 in place of the external manipulation section 12′ for example. In such an arrangement, instead of gripping a pulling member upstream of a sheath 32, the sheath 32 itself could be gripped between the cam cleats 122, 126 and the ledges 124, 128. To accommodate a sheath 32 instead of a pulling member 118, the distance between each cam cleat 122, 126 and its respective ledges 124, 128 may need to be altered.

Furthermore, the described deployment handle 100 could be used to move or withdraw other components of an implant deployment device 10. For example, it could be used to withdraw restraining wires, such as distal restraining wire 42 shown in FIGS. 1 and 2, that hold an implant 18 in a constrained configuration.

In a modification, the pulling member 118, sheath 32 or other component of an implant deployment device may include a profiled surface (for example a plurality of ridges or teeth arranged therealong). The teeth on the gripping surface of the cam cleats 122, 126 can then interengage with the profiled surface of the component to be withdrawn.

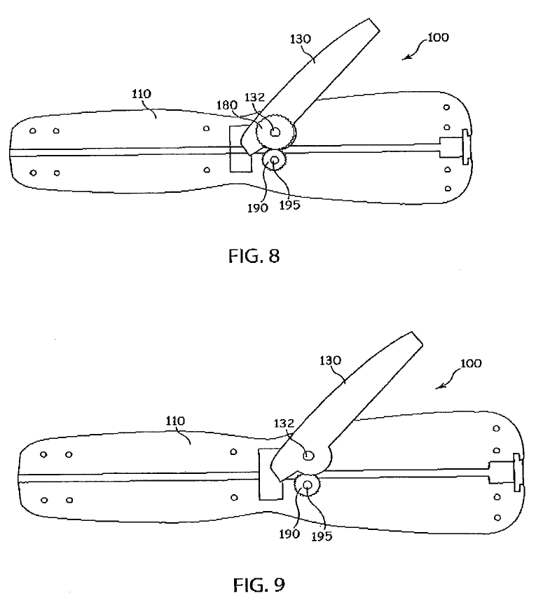

FIGS. 8 and 9 illustrate another embodiment of a deployment handle 100. Both Figures show cross-sections through the housing 110, but in FIG. 8, the outlines of all components are shown so their relative arrangements can be seen.

Extending from within the housing 110 is a lever 130. The lever 130 is mounted within the housing by a lever pivot 132. Surrounding the lever pivot 132 is a first toothed wheel 180. Mounted to the inside of the housing by means of a pin 195, adjacent the first toothed wheel 132, is a second toothed wheel 190. The first toothed wheel 180 is fixed to the lever 130 so that relative rotation between the first toothed wheel 180 and the lever 130 is prevented. The second toothed wheel 190 is able to rotate around the pin 195. The teeth of the first toothed wheel 180 and the second toothed wheel 190 are able to mesh.

A component to be withdrawn, for example a sheath 32 is threaded longitudinally through the housing 110 and between the first toothed wheel 180 and the second gear wheel 190. In use, squeezing the lever 130 towards the housing 110 causes clockwise rotation of the toothed wheel 180 (in the view shown in FIGS. 8 and 9). As the toothed wheel 180 rotates clockwise, the second toothed wheel 190 rotates anticlockwise, and the component to be moved is pulled through the toothed wheels (towards the left-hand side of the drawings shown in FIGS. 8 and 9).

It will be appreciated that many modifications could be made to the embodiments shown in FIGS. 8 and 9. For example, the skilled person would appreciate that the first toothed wheel 180 could be replaced by a one-way ratchet wheel. This would allow the lever 130 to be lifted without causing movement of the component back in the direction opposite to the withdrawal direction. In this way, greater control can be achieved by the use of several lever squeezes to provide the desired extend of withdrawal. The lever 130 may be biased into its raised position. The skilled person will also appreciate that preferred features and modifications described in respect of the embodiment illustrated in FIGS. 3 to 7 can be applied to the embodiment shown in FIGS. 8 and 9 as appropriate.

Other uses of the disclosed deployment handles will be envisaged by the skilled person.

The disclosures in U.S. patent application No. U.S. 60/998,098, from which this application claims priority, and in the abstract accompanying this application are incorporated herein by reference.

What is claimed is:

1. A deployment handle for moving a component of an implant deployment device, including:
a withdrawal member able to be coupled to a component to be moved, and
an actuator able to move towards a withdrawal axis of the handle,
wherein movement of the actuator in a direction towards the axis actuates the withdrawal member, thereby to move a component in a withdrawal direction.

2. The deployment handle of claim 1, wherein the withdrawal member is moveable and movement of the actuator in a direction towards the axis results in movement of the withdrawal member in a withdrawal direction.

3. The deployment handle of claim 2, wherein movement of the actuator in a direction away from the axis results in movement of the withdrawal member in a direction opposite to the withdrawal direction.

4. The deployment handle of claim 1, wherein the withdrawal member is able to move in a withdrawal direction from a first position to a second position, and wherein the withdrawal member is biased into its first position.

5. The deployment handle of claim 1, wherein the actuator has an actuation position, wherein it is towards the axis, and a release position, wherein it is away from the axis, and wherein the actuator is biased into the release position.

6. The deployment handle of claim 4, wherein the withdrawal member and the actuator are arranged such that the withdrawal member is biased into its first position and the actuator is biased into its release position.

7. The deployment handle of claim 1, wherein the withdrawal member includes a first gripping element.

8. The deployment handle of claim 7, wherein the withdrawal member is moveable and wherein the first gripping element is operable to grip a component during movement of the moveable withdrawal member in a withdrawal direction and to release its grip on a component during movement of the withdrawal member in a direction opposite to the withdrawal direction.

9. The deployment handle of claim 7, wherein the first gripping element is arranged to grip a component to be moved when relative movement between the component and the first gripping element is in a first direction, and wherein the first gripping element is arranged to release the component to be moved when relative movement between the component and the first gripping element is in a second direction, movement of the first gripping element whilst gripping a component to be moved thereby resulting in movement of the component.

10. A deployment handle for moving a component of an implant deployment device, including:
a moveable withdrawal member and a first gripping element for gripping a component to be moved, wherein the first gripping element is provided on the moveable withdrawal member, and wherein the first gripping element is operable to grip a component during movement of the moveable withdrawal member in a withdrawal direction and to release its grip on a component during movement of the withdrawal member in a direction opposite to the withdrawal direction.

11. The deployment handle of claim 10, wherein the first gripping element is arranged to grip a component to be moved when relative movement between a component and the gripping element is in a first direction, and wherein the first gripping element is arranged to release a component to be moved when relative movement between a component and the gripping element is in a second direction, movement of the gripping element whilst gripping a component to be moved thereby resulting in movement of a component.

12. The deployment handle of claim 11, wherein the first gripping element is biased into a gripping state.

13. The deployment handle of claim 11, including a second gripping element for gripping a component to be moved, wherein the second gripping element is operable to grip a component to be moved during release of the component to be moved by the first gripping element.

14. The deployment handle of claim 11, including a second gripping element for gripping a component to be moved, wherein the second gripping element is arranged to grip a component to be moved when relative movement between the component and the second gripping element is in a first direction, and wherein the second gripping element is arranged to release the component to be moved when relative movement between the component and the second gripping element is in a second direction.

15. The deployment handle of claim 13, wherein the second gripping element is biased into a gripping state.

16. The deployment handle of claim 11, wherein the first gripping element and/or the second gripping element includes a cam cleat and an opposed ledge between which a component may be gripped.

17. The deployment handle of claim 11, including an actuator able to move towards a withdrawal axis of the handle, wherein movement of the actuator in a direction towards the axis actuates the withdrawal member, thereby to move a component in a withdrawal direction.

18. A deployment handle for withdrawing a component of an implant deployment device, including:
a moveable withdrawal member able to be coupled to a component to be withdrawn;
a first gripping element for gripping a component to be withdrawn, wherein the first gripping element is provided on the moveable withdrawal member, and wherein the first gripping element is operable to grip a component during movement of the moveable withdrawal member in a withdrawal direction and to release its grip on a component during movement of the withdrawal member in a direction opposite to the withdrawal direction, wherein the first gripping element is biased into a gripping state;
a second gripping element for gripping a component to be moved, wherein the second gripping element is operable to grip a component to be moved during release of the component to be moved by the first gripping element, wherein the second gripping element is biased into a gripping state;
an actuator able to move towards a withdrawal axis of the handle, wherein movement of the actuator in a direction towards the axis actuates the withdrawal member resulting in movement of the withdrawal member in a withdrawal direction from a first position to a second position, thereby to withdraw a component, wherein the actuator has an actuation position, wherein it is towards the axis, and a release position, wherein it is away from the axis, and wherein the actuator is biased into the release position and wherein the withdrawal member is biased into its first position.

19. A kit including an implant deployment device including a component to be moved, and the deployment handle of claim 1.

20. The kit of claim 19, wherein the deployment handle includes a gripping element including a ridged gripping surface and wherein the component to be moved includes a ridged surface to engage with the ridged gripping surface of the gripping element.
